(19) United States
(12) Patent Application Publication
(10) Pub. No.: US 2003/0175353 A1
(43) Pub. Date: Sep. 18, 2003

(54) Oral controlled drug delivery system

(75) Inventors: Kamlesh Mohanlal Dudhara, Baroda (IN); Ashish Prabhakar Mungre, Mumbai (IN)

Correspondence Address: ARMSTRONG,WESTERMAN & HATTORI, LLP, 1725 K STREET, NW, SUITE 1000, WASHINGTON, DC, 20006, US

(73) Assignee: SUN PHARMACEUTICAL INDUSTRIES LIMITED, Mumbai (IN)

(21) Appl. No.: 10/387,560

(22) Filed: Mar. 14, 2003

(30) Foreign Application Priority Data
Mar. 14, 2002 (IN) 249/MUM/2002

(51) Int. Cl. A61K 31/55; A61K 9/14

(52) U.S. Cl. 424/486; 514/217

(57) ABSTRACT

The oral controlled drug delivery system of the present invention comprises carbamazepine and one or more hydrophobic polymers in homogenous admixture, wherein the system does not comprise any means capable of preventing the conversion of carbamazepine to its dihydrate form. The present invention provides an oral controlled drug delivery system for carbamazepine having a desirable controlled rate of delivery of carbamazepine, which system is simple, uncomplicated and easy to manufacture.

### FIELD OF THE INVENTION

The present invention relates to an oral controlled drug delivery system for carbamazepine.

### BACKGROUND OF THE INVENTION

Carbamazepine, 5H-dibenz-[b,f]azepine-5-carboxamide, is used as an anti-convulsant and is available commercially in the form of tablets, syrups, chewable tablets and extended-release formulations. It is used in epileptic patients who do not respond satisfactorily to other forms of treatment. The drug appears to act by reducing polysynaptic responses and by blocking post-tetanic potentiation.

The therapeutically effective blood levels of carbamazepine are from about 4 μg/ml to about 12 μg/ml. Blood levels of carbamazepine below 4 μg/ml are ineffective in treating clinical disorders, while levels above 12 μg/ml are most likely to result in side-effects. The side effects are seen to a greater extent in syrup formulations due to the presence of fine particles of the active ingredient, which dissolve rapidly leading to faster drug absorption and higher peak plasma levels. The tablet formulations are relatively free of this disadvantage.

Modified release formulations of carbamazepine produce substantial reduction in intra-dose fluctuations in carbamazepine concentrations, and thus tolerability and seizure control in patients with epilepsy may be improved (Martindale—The Complete Drug Reference; 32^{nd} edition, ed. by Kathleen Parfitt, Pharmaceutical Press, 1999).

The oral osmotic system (OROS®, Alza Corp.) described by F. Thecuwes in J. Pharm. Sci., Vol. 64, 12, 1987-1991 (1975) and in U.S. Pat. No. RE 34990 comprises a core comprising carbamazepine and a protective colloid, a semi-permeable wall covering the drug-containing core and a passageway through the wall. Water permeates from the surrounding body fluids through the semi-permeable wall and the pressure that is built-up causes a suspension of the drug to be released from the passageway. A problem encountered with the osmotic system for carbamazepine was that when fine particles of anhydrous carbamazepine were used, upon contact with water large needles of the dihydrate form of carbamazepine were formed. These crystals blocked the orifice of the osmotic system. Thus, osmotic system of RE 34990 used hydroxypropyl methylcellulose as a protective colloid to prevent the conversion of the fine particles of anhydrous carbamazepine to the dihydrate form. However, even with the resolution of this problem the osmotic system has other disadvantages. One concern is that the desired dose of carbamazepine should be pushed out of the osmotic system and become absorbed. The manufacture of oral osmotic drug delivery systems is complicated, involving procedures such as organic solvent based coating to form the semi-permeable membrane, and formation of the orifice or passageway using mechanical or laser drilling techniques. Particularly, the use of organic solvent based coatings is undesirable due to environmental, safety and cost considerations.

U.S. Pat. No. 5,326,570 claims a drug delivery system comprising a combination of an immediate release, a sustained release and an enteric release unit containing carbamazepine. The patent does not disclose or exemplify drug delivery system comprising hydrophobic polymers for desired controlled delivery of carbamazepine.

U.S. Pat. No. 5,980,942 claims an erodible oral composition for sustained delivery of a drug at a zero order release rate comprising a pharmaceutical agent in combination with an erodible polymer matrix, comprising at least one hydrophilic polymer or a mixture of two or more hydrophilic polymers having a molecular weight between 10,000 and 246,000; wherein the matrix inhibits the conversion of carbamazepine to the dihydrate form. The erodible polymer matrix of the system is thus hydrophilic in nature and may optionally contain a hydrophobic component. The system does not disclose an oral controlled drug delivery system capable of delivering carbamazepine at a desirable controlled rate of delivery in the absence of a protective colloid that inhibits the conversion of anhydrous carbamazepine to its dihydrate form.

U.S. Pat. No. 6,162,466 claims a tablet for a prolonged release of carbamazepine, comprising (a) a pharmaceutically effective amount of carbamazepine particles, (b) a methacrylic polymer, and (c) additional excipients, wherein said methacrylic polymer and at least one additional excipient form a single coating layer over said carbamazepine particles and at least one additional excipient is present as an extragranular ingredient. The patent discloses a system wherein a hydrophobic coating dispersion of the methacrylic polymer containing a hydrophobic plasticiser is used to form a single coating layer on carbamazepine particles, which are then blended with other excipients and compressed. The disclosure in the patent particularly points to the need to provide a single layer coating over the carbamazepine particles with methacrylic polymers. Moreover, only particular water insoluble grades of methacrylic acid polymer are exemplified.

### OBJECT OF THE INVENTION

It is an object of the present invention to provide an oral controlled drug delivery system for carbamazepine.

It is a further object of the present invention to provide a simple, uncomplicated and easy to manufacture oral controlled drug delivery system for desirable controlled rate of delivery of carbamazepine.

It is still a further object of the present invention to provide an oral controlled drug delivery system capable of delivering carbamazepine at a desirable controlled rate wherein anhydrous carbamazepine is allowed to convert to needle-shaped crystals of its dihydrate form. Thus, the system may be formulated without a particular requirement for a means for preventing conversion of the anhydrous carbamazepine upon its contact with water to large needle-shaped crystals of its dihydrate form.

It is also an object of the present invention to provide a simple, uncomplicated and easy to manufacture oral controlled drug delivery system that is capable of delivering carbamazepine at a desirable controlled rate of delivery, such that the system is bioequivalent to marketed carbamazepine controlled drug delivery systems that release carbamazepine in a controlled zero order manner.

### SUMMARY OF THE INVENTION

The oral controlled drug delivery system of the present invention comprises carbamazepine and one or more hydrophobic polymers in homogenous admixture, wherein the system does not comprise any means capable of preventing the conversion of carbamazepine to its dihydrate form. The present invention provides an oral controlled drug delivery system for carbamazepine having a desirable controlled rate of delivery of carbamazepine, which system is simple, uncomplicated and easy to manufacture. The present invention provides an oral controlled drug delivery system that is bioequivalent with osmotic controlled zero-order carbamazepine drug delivery system commercially available in the United States of America.

### DETAILED DESCRIPTION OF THE INVENTION

The present invention provides an oral controlled drug delivery system for carbamazepine having a desirable controlled rate of delivery of carbamazepine, which system is simple, uncomplicated and easy to manufacture.

The present invention comprises an oral controlled drug delivery system comprising carbamazepine and one or more hydrophobic polymers in homogenous admixture, wherein the system does not comprise any means capable of preventing the conversion of carbamazepine to its dihydrate form.

The homogenous admixture of carbamazepine and one or more hydrophobic polymers, optionally with other pharmaceutically acceptable excipients, may be in the form of granules, pellets, extrudates, tablets and other forms well known to those skilled in the art. The oral controlled drug delivery system of the present invention is formed by a simple, uncomplicated and easy to manufacture process involving the steps of mixing and compressing.

The carbamazepine used in the present invention may be carbamazepine in crystalline or amorphous form, and it may be anhydrous carbamazepine or a hydrate of carbamazepine. Preferably, the carbamazepine used is crystalline anhydrous carbamazepine. The carbamazepine used may be milled carbamazepine, or micronised carbamazepine, or a mixture of milled carbamazepine and micronised carbamazepine. The milled and micronised fractions of carbamazepine may be mixed in a ratio so that the particle size distribution of the mixture is such that at least 90% of the particles are below 50 microns. In preferred embodiments, the milled carbamazepine is mixed with the micronised carbamazepine in a ratio of 3:1 such that the mean diameter of the particles of the mixture is about 21 microns.

In prior art protective colloids have been used for preventing the conversion of anhydrous carbamazepine upon its contact with water to large needle-shaped crystals of its dihydrate form. For example, U.S. Pat. No. RE 34990 and U.S. Pat. No. 5,284,662 disclose the use of protective colloids selected from the group consisting of C₁₋₄alkyl cellulose, hydroxypropyl-C₁₋₄alkyl cellulose, sodium carboxymethyl cellulose, sodium carboxymethyl C₁₄ alkyl cellulose, and gelatin, for preventing the conversion of anhydrous carbamazepine upon its contact with water to large needle-shaped crystals of its dihydrate form. Other prior arts disclose crystal habit modifiers that allow the conversion of anhydrous carbamazepine to the dihydrate form. However, the dihydrate form is not in the form of large needle-shaped crystals. For example, U.S. Pat. No. 5,122,543 discloses the use of vinylpyrrolidone/vinyl acetate copolymer as crystal habit modifier. U.S. Pat. No. 6,534,090, corresponding to Indian Patent Application No. 119/MUM/2001, discloses an oral osmotic controlled drug delivery system, wherein in the presence of a crystal habit modifier, the anhydrous carbamazepine is converted to cuboidal and/or rod-shaped crystals of the dihydrate form, thus restricting the formation of needle-shaped crystals. Crystal habit modifiers useful for the purpose included vinylpyrrolidone polymers, vinylpyrrolidone/vinyl acetate polymers, polyethylene oxide polymers, polyoxyethylene-polyoxypropylene glycol copolymers, polyoxyethylene castor oil derivatives, long chain C₁₂-C₁₈ fatty acid glycerides and mixtures thereof. The oral controlled drug delivery system of the present invention is capable of delivering carbamazepine at a desirable controlled rate of delivery without a particular requirement for preventing the conversion of carbamazepine, upon its contact with water, to large needle-shaped crystals.

Hydrophobic polymers well known to those skilled in the art may be used to prepare the oral controlled drug delivery system of the present invention. Examples of hydrophobic polymers that may be used in the present invention include insoluble polymers such as fine powders of ammoniomethacrylate copolymers, latex dispersions of methacrylic ester copolymers, hydrophobic polyacrylamide derivatives, cellulose derivatives such as ethylcellulose, cellulose acetate, cellulose acetate butyrate, cellulose acetate propionate, and the like; stearyl alcohol, low molecular weight polyethylene, glyceryl palmitostearate, glyceryl monostearate, waxes such as carnauba wax, beeswax, candelilla wax, microcrystalline wax, ozokerite wax, paraffin waxes, castorwax (hydrogenated castor oil); and mixtures thereof.

In a preferred embodiment of the present invention the hydrophobic polymer is a cellulose ether, preferably ethylcellulose. Ethylcellulose is an ethyl ether of cellulose, with a long-chain polymer of β-anhydroglucose units joined together by acetal linkages and is commercially available in different grades, the grades being based on the viscosity provided by the solution of ethylcellulose. In the present invention, the ethylcellulose is selected such that the viscosity of a 5% solution of ethylcellulose in a mixture of toluene and ethanol is in the range of about 40 mPas to about 100 mPas, preferably from about 40 mPas to about 60 mPas. Various commercial brands of ethylcellulose that provide these characteristics are available and can be used in the present invention. In a preferred embodiment, the ethylcellulose used is Ethocel Standard 45 Premium, a 5% solution of the same in a mixture of 80% toluene and 20% ethanol having a viscosity of 41-49 mPas. It is used in an amount ranging from about 2% to about 50% by weight of the system, preferably from about 2% to about 10% by weight of the system, more preferably from about 2% to about 5% by weight of the system.

The oral controlled drug delivery system of the present invention may further include hydrophilic polymers. Hydrophilic polymers known to those skilled in the art and those hydrophilic polymers that do not act as protective colloid that effectively prevent conversion of carbamazepine to its dihydrate form upon contact with water may be used in the present invention. In preferred embodiments the hydrophilic polymer used in a vinyl pyrrolidone polymer.

Vinyl pyrrolidone polymers or polyvinylpyrrolidone (PVP), also referred to as Povidone, are synthetic polymers consisting essentially of linear 1-vinyl-2-pyrrolidinone groups, the degree of polymerization of which results in polymers of various molecular weights, the molecular weight ranging between 2500 and 3,000,000 Daltons. PVP is commercially available as Kollidone (BASF), Plasdone® and Peristone® (General Aniline). PVP is classified into different grades on the basis of its viscosity in aqueous solution. Different grades of PVP available are PVP K-12, PVP K-15, PVP K-17, PVP K-25, PVP K-30, PVP K-60, PVP K-90 and PVP K-120. The K-value referred to in the above nomenclature is calculated from the viscosity of the PVP in aqueous solution, relative to that of water. In preferred embodiments the PVP used is PVP K-30 having an approximate molecular weight of 50,000 Daltons. It is used in an amount ranging from about 0.5% to about 10% by weight of the system, more preferably from about 1% to about 5% by weight of the system.

The oral controlled drug delivery system of the present invention may include a wicking agent such as microcrystalline cellulose. Microcrystalline cellulose (MCC) is made up of a chain of about 250 glucose molecules in the form of a microcrystal, consisting primarily of crystallite aggregates obtained by removing amorphous regions of a pure cellulose source material by hydrolytic degradation using mineral acid. MCC has an average molecular weight of about 36,000 Daltons and is available in various grades, which differ in bulk density, particle size and moisture content. It is commercially available as Vivapur®, Avicel®, Vivacel®, Emcocel®, Fibrocel® and Tabulose®. In preferred embodiments, the microcrystalline cellulose used is Avicel® PH 101 having a typical average particle size of 50 μm, a bulk density of 0.28 g/cc and loss on drying of 4%. It is used in an amount ranging from about 0.5% to about 40% by weight of the system, preferably from about 2% to about 20% by weight of the system.

The oral controlled drug delivery system of the present invention may also include various pharmaceutically acceptable excipients, for example disintegrants such as starch, cellulose derivatives, gums, crosslinked polymers and the like; binders such as starch, gelatin, sugars, cellulose derivatives, polyvinylpyrrolidone and the like; and lubricants. In preferred embodiments, starch is used as the disintegrant in an amount ranging from about 0.5% to about 2% by weight of the system. In other embodiments, the system may further include croscarmellose sodium in admixture with starch, as the disintegrant. Croscarmellose sodium may be used in an amount ranging from about 0.5% to about 5% by weight of the system.

Examples of lubricants that may be used in the present invention include talc, magnesium stearate, calcium stearate, aluminum stearate, stearic acid, hydrogenated vegetable oils, colloidal silicon dioxide, polyethylene glycol, cellulose derivatives such as carboxyalkyl cellulose and its alkali salts, or mixtures thereof. Preferred embodiments use a combination of colloidal silicon dioxide, croscarmellose sodium, magnesium stearate and talc as a lubricant, the combination being used in an amount ranging from about 0.5% to about 5% by weight of the system. Colloidal silicon dioxide is available commercially as Aerosil® from Degussa-Huls, Nippon and Fischer GmbH. Croscarmellose sodium is a crosslinked polymer of sodium carboxymethyl cellulose, also known as Ac-Di-Sol, and available commercially as Nymeel® ZSX, Pharmacel® XL, Primellose® or Solutab®.

In a preferred embodiment, the oral controlled drug delivery system comprises (a) crystalline anhydrous carbamazepine having a particle size distribution such that at least 90% of the particles are below 50 microns, in an amount ranging from about 60% to about 85% by weight of the system; (b) ethylcellulose, selected such that a 5% solution of the same in a mixture of toluene and ethanol has a viscosity of 40-60 mPas, in an amount ranging from about 3% to about 10% by weight of the system; (c) vinyl pyrrolidone polymer having an approximate molecular weight of 50,000, in an amount ranging from about 1% to about 5% by weight of the system; (d) microcrystalline cellulose in an amount ranging from about 5% to about 25% by weight of the system; (e) starch, in an amount ranging from about 0.5% to about 2% by weight of the system; and (f) croscarmellose sodium in an amount ranging from about 0.5% to about 5% by weight of the system.

In preferred embodiments, the oral controlled drug delivery system is in the form of tablets that disintegrate in the gastric fluid. The oral controlled drug delivery system of the present invention may optionally be covered with a water-soluble polymer that does not function as a release rate controlling polymer. For example, the system comprising carbamazepine may be coated with a film of a cationic polymer based on dimethylaminoethyl methacrylate and other neutral methacrylic acid esters, particularly poly (butyl methacrylate, (2-dimethyl aminoethyl) methacrylate, methyl methacrylate) (1:2:1), available worldwide under the brandname Eudragit E 100. Eudragit E 100 provides a film that is soluble in gastric fluid below pH 5, and is not a release rate controlling polymer.

The present invention provides an oral controlled drug delivery system that releases carbamazepine in a controlled manner to provide desirable blood level profile of carbamazepine that minimizes side effects, while providing efficacy. For example, when administered as a single dose in fasted state to healthy human subjects it provides area under the plasma concentration-time curve (AUC) which is comparable to that provided by the oral osmotic controlled zero-order drug delivery system commercially available in the United States of America. Alternatively, it provides peak plasma levels (Cₘₐₓ) that are comparable with those provided by the oral osmotic controlled zero-order drug delivery system commercially available in the United States of America. Herein, the term capable means that 90 percent confidence intervals for the ratio of the population geometric means between the oral controlled drug delivery system of the present invention and the oral osmotic controlled zero-order drug delivery system commercially available in the United States of America, namely Tegretol XR®, based on log-transformed data, is contained in the limits of 70-135 percent for AUC and Cₘₐₓ. More preferred embodiments of the present invention are bioequivalent to marketed carbamazepine controlled drug delivery systems that release carbamazepine in a controlled zero order manner. Bioequivalence may be determined according to United States Food and Drug Administration (USFDA) guidelines and criteria.

One embodiment of the present invention provides an oral controlled drug delivery system comprising 200 mg carbamazepine, one or more hydrophobic polymers and optionally pharmaceutically acceptable excipients, such that the system upon oral administration to healthy male volunteers gives a plasma concentration versus time profile with mean plasma concentration lying in the concentration ranges as given below—

| Time (hours) | Plasma concentration range (μg/ml) |
|---|---|
| 8.0 | 1.1-2.0 |
| 20.0 | 1.4-2.5 |
| 48.0 | 1.0-1.8 |
| 120.0 | 0.3-0.7 |

In one embodiment, the present invention provides an oral controlled drug delivery system comprising 400 mg carbamazepine, one or more hydrophobic polymers and optionally pharmaceutically acceptable excipients, such that the system upon oral administration to healthy male volunteers gives a plasma concentration versus time profile with mean plasma concentration lying in the concentration ranges as given below—

| Time (hours) | Plasma concentration range (μg/ml) |
|---|---|
| 8.0 | 0.9-3.3 |
| 20.0 | 2.4-4.4 |
| 48.0 | 2.0-3.4 |
| 120.0 | 0.6-1.0 |

A process for the preparation of the oral controlled drug delivery system of the present invention comprises mixing the carbamazepine, hydrophobic polymers and pharmaceutically acceptable excipients to obtain a dry powder blend. A hydrophilic polymer, when present, is mixed with this dry powder blend. The dry powder blend is then granulated by conventional means. The granules may be filled into capsules, or preferably compressed into tablets. Alternatively, the dry powder blend may be directly compressed into tablets A solution of the film former may then be used to coat the tablets thus obtained, using methods known to a person skilled in the art.

The examples that follow do not limit the scope of the invention and are included as illustrations.

### EXAMPLE 1

The oral controlled release tablets of the present invention were prepared as per Table 1 below.

TABLE 1

| Ingredients | Quantity (mg/tablet) | Percent (%) by weight |
|---|---|---|
| Carbamazepine | 200.0 | 72.72 |
| Ethylcellulose (Ethocel | 11.50 | 4.18 |
| Standard 45 Premium) | | |
| Microcrystalline cellulose | 33.75 | 12.27 |
| (Avicel ® PH 101) | | |
| Starch | 2.00 | 0.73 |
| Talc | 10.5 | 3.82 |
| Magnesium stearate | 2.50 | 0.90 |
| Colloidal silicon dioxide | 2.50 | 0.90 |
| Croscarmellose sodium | 2.50 | 0.90 |
| Coat | | |
| Eudragit E 100* | 2.50 | 0.90 |
| Talc | 3.15 | 1.14 |
| Magnesium stearate | 1.00 | 0.36 |
| Titanium dioxide | 2.00 | 0.73 |
| Red oxide of Iron | 0.10 | 0.03 |
| Iron oxide yellow | 0.25 | 0.09 |
| Polyethylene glycol 6000 | 0.50 | 0.18 |

*-poly (butyl methacrylate, (2-dimethyl aminoethyl) methacrylate, methyl methacrylate) 1:2:1

Carbamazepine and a part of the ethylcellulose were milled separately and sifted. Microcrystalline cellulose and starch were sifted through ASTM (American Society for Testing and Materials) #60 sieve. All the milled and sifted ingredients were mixed to obtain a dry powder blend. The binder solution was obtained by dissolving the remaining quantity of ethylcellulose in isopropyl alcohol. The binder solution was added to the dry powder blend and granulated in a rapid mixer granulator. The granules thus obtained were wet milled, dried in a fluid bed dryer and sifted through ASTM #20 sieve. The granules were then subjected to dry milling. Talc, magnesium stearate, colloidal silicon dioxide and croscarmellose sodium were sifted separately and used to lubricate the milled granules. This was followed by compression of the lubricated mass to obtain the tablet. The coating solution was prepared by mixing a first solution of Eudragit E 100 in isopropyl alcohol and acetone, a second solution of talc, magnesium stearate, titanium dioxide, red oxide of iron and iron oxide yellow in isopropyl alcohol, and a third solution of polyethylene glycol 6000 in water. The coating solution thus obtained was used to coat the tablet. The controlled release tablets thus obtained were subjected to in vitro dissolution testing using United States Pharmacopoeia Type I dissolution apparatus at 100 rpm. The dissolution medium used was 900 ml of purified water. The results obtained are recorded in Table 2 below.

TABLE 2

| Time (hours) | % drug released |
|---|---|
| 3 | 39 |
| 6 | 52 |
| 12 | 63 |
| 24 | 76 |

The bioavailability of the oral controlled drug delivery system for carbamazepine (Example 1) and that of marketed carbamazepine controlled drug delivery systems were studied. A single dose, open label, randomized, comparative, two-way crossover study was carried out for the same. Tegretol® XR 200 mg tablets (Novartis, Lot No. IT246507) were used as the reference standard.

The pharmacokinetic assessment was based on the plasma levels of carbamazepine measured by blood sampling. Blood samples were obtained before dosing and at the following times after administration of both the reference and test medications—1, 2, 4, 6, 8, 10, 12, 14, 16, 18, 20, 24, 28, 32, 36, 48, 72, 96, 120, 144 and 196 hours.

Eleven healthy male volunteers were enrolled for the study and all of them completed the two-way crossover study. The subjects were fasted overnight before dosing and for 4 hours thereafter. Drinking water was prohibited 2 hours before dosing and 2 hours thereafter, but was allowed ad lib at all other times. Standard meals were provided at 4, 12, 28 and 36 hours after dosing and at appropriate times thereafter. Meal plans were identical for both the periods.

Subjects received a single tablet of carbamazepine (200 mg, Example 1) with 240 ml of water at ambient temperature after the overnight fast, as the test medication, while a single tablet of Tegretol® XR 200 mg (Novartis) was administered as the reference medication.

The plasma concentration of carbamazepine was determined for samples collected at different time points and averaged over the eleven volunteers. The data is given in Table 3 below.

TABLE 3

Plasma concentration (ng/ml) (Mean ± SD)

| | Carbamazepine tablet | Tegretol ® XR 200 mg |
|---|---|---|
| Time (hrs) | (Example 1) | (Novartis) |
| 0 | 0 | 0 |
| 1.0 | 342.69 ± 149.80 | 72.00 ± 203.29 |
| 2.0 | 598.39 ± 249.99 | 412.06 ± 319.65 |
| 4.0 | 1229.71 ± 299.35 | 962.21 ± 209.75 |
| 6.0 | 1460.74 ± 334.49 | 1385.34 ± 162.69 |
| 8.0 | 1569.64 ± 318.59 | 1564.67 ± 231.98 |
| 10.0 | 1737.42 ± 319.00 | 1750.93 ± 313.29 |
| 12.0 | 1810.64 ± 376.12 | 1788.40 ± 378.10 |
| 14.0 | 1985.70 ± 328.68 | 1850.08 ± 361.85 |
| 16.0 | 1926.95 ± 296.79 | 1895.27 ± 342.66 |
| 18.0 | 1895.30 ± 284.97 | 1877.66 ± 282.27 |
| 20.0 | 1886.38 ± 317.56 | 2023.65 ± 338.13 |
| 24.0 | 2069.90 ± 413.74 | 1840.12 ± 276.51 |
| 28.0 | 1953.97 ± 328.05 | 1811.48 ± 354.99 |
| 32.0 | 1939.41 ± 156.14 | 1782.77 ± 306.98 |
| 36.0 | 1810.75 ± 277.79 | 1652.47 ± 290.34 |
| 48.0 | 1558.01 ± 335.06 | 1402.71 ± 258.80 |
| 72.0 | 1256.84 ± 306.06 | 1027.01 ± 168.16 |
| 96.0 | 736.57 ± 256.13 | 686.71 ± 117.29 |
| 120.0 | 539.44 ± 212.52 | 499.06 ± 123.78 |
| 144.0 | 393.97 ± 214.22 | 325.47 ± 117.91 |
| 196.0 | 232.30 ± 176.71 | 163.84 ± 101.00 |

The composition of the present invention was found to be bioequivalent to Tegretol® XR, an osmotic system that releases carbamazepine in a controlled zero order manner.

### Example 3

Another embodiment of the oral controlled release tablet of carbamazepine was prepared as per Table 4 below.

TABLE 4

| Ingredients | Quantity (mg/tablet) | Percent (%) by weight |
|---|---|---|
| Carbamazepine (Milled) | 400.00 | 74.14 |
| Ethyl Cellulose (Ethocel std | 23.00 | 4.26 |
| 45 premium) | | |
| Microcrystalline Cellulose | 67.00 | 12.42 |
| (Avicel PH101) | | |
| Starch | 4.00 | 0.74 |
| Croscarmellose Sodium | 5.00 | 0.93 |
| (Ac-di-sol) | | |
| Colloidal Silicon dioxide | 5.00 | 0.93 |
| Talc | 21.00 | 3.89 |
| Magnesium Stearate | 5.00 | 0.93 |
| Coat | | |
| Eudragit E 100* | 2.50 | 0.46 |
| Talc | 3.15 | 0.58 |
| Magnesium stearate | 1.00 | 0.18 |
| Titanium dioxide | 2.00 | 0.37 |
| Red oxide of Iron | 0.10 | 0.018 |
| Iron oxide yellow | 0.25 | 0.046 |
| Polyethylene glycol 6000 | 0.50 | 0.093 |

*-poly (butyl methacrylate, (2-dimethyl aminoethyl) methacrylate, methyl methacrylate) 1:2:1

The tablets were obtained as per the method given in Example 1 above.

The tablets thus obtained were subjected to in vitro dissolution testing using United States Pharmacopoeia Type I dissolution apparatus at 100 rpm. The dissolution medium used was 2000 ml or purified water with 0.1% hydroxypropyl methylcellulose. The results obtained are recorded in Table 5 below.

TABLE 5

| Time (hours) | % drug released |
|---|---|
| 1 | 23 |
| 3 | 42 |
| 6 | 57 |
| 12 | 72 |
| 16 | 78 |
| 24 | 83 |

### EXAMPLE 4

The oral controlled release tablets of carbamazepine were prepared as per Table 6 below.

TABLE 6

| Ingredients | Quantity (mg/tablet) | Percent (%) by weight |
|---|---|---|
| Carbamazepine | 400.00 | 74.14 |
| (Micronised) | | |
| Ethyl Cellulose (Ethocel std | 23.00 | 4.26 |
| 45 premium) | | |
| Microcrystalline Cellulose | 67.0 | 12.42 |
| (Avicel PH101) | | |
| Starch | 4.00 | 0.74 |
| Croscarmellose Sodium | 5.00 | 0.93 |
| (Ac-di-sol) | | |
| Colloidal Silicon dioxide | 5.00 | 0.93 |
| Talc | 21.00 | 3.89 |
| Magnesium Stearate | 5.00 | 0.93 |
| Coat | | |
| Eudragit E 100* | 2.50 | 0.46 |
| Talc | 3.15 | 0.58 |
| Magnesium stearate | 1.00 | 0.18 |
| Titanium dioxide | 2.00 | 0.37 |
| Red oxide of Iron | 0.10 | 0.018 |
| Iron oxide yellow | 0.25 | 0.046 |
| Polyethylene glycol 6000 | 0.50 | 0.093 |

*-poly (butyl methacrylate, (2-dimethyl aminoethyl) methacrylate, methyl methacrylate) 1:2:1

The tablets were obtained as per the method given in Example 1 above.

The tablets thus obtained were subjected to in vitro dissolution testing using United States Pharmacopoeia Type I dissolution apparatus at 100 rpm. The dissolution medium used was 2000 ml of purified water with 0.1% hydroxypropyl methylcellulose. The results obtained are recorded in Table 7 below.

TABLE 7

| Time (hours) | % drug released |
|---|---|
| 1 | 25 |
| 3 | 45 |
| 6 | 59 |
| 12 | 73 |
| 16 | 79 |
| 24 | 86 |

### EXAMPLE 5

The oral controlled release tablets of carbamazepine were prepared as per Table 8 below.

TABLE 8

| Ingredients | Quantity (mg/tablet) | Percent (%) by weight |
|---|---|---|
| Carbamazepine (Milled) | 300.00 | 55.61 |
| Carbamazepine | 100.00 | 18.53 |
| (Micronised) | | |
| Ethyl Cellulose (Ethocel std | 23.00 | 4.26 |
| 45 premium) | | |
| Microcrystalline Cellulose | 67.0 | 12.42 |
| (Avicel PH101) | | |
| Starch | 4.00 | 0.74 |
| Croscarmellose Sodium | 5.00 | 0.93 |
| (Ac-di-sol) | | |
| Colloidal Silicon dioxide | 5.00 | 0.93 |
| Talc | 21.00 | 3.89 |
| Magnesium Stearate | 5.00 | 0.93 |
| Coat | | |
| Eudragit E 100* | 2.50 | 0.46 |
| Talc | 3.15 | 0.58 |
| Magnesium stearate | 1.00 | 0.18 |
| Titanium dioxide | 2.00 | 0.37 |
| Red oxide of Iron | 0.10 | 0.018 |
| Iron oxide yellow | 0.25 | 0.046 |
| Polyethylene glycol 6000 | 0.50 | 0.093 |

*-poly (butyl methacrylate, (2-dimethyl aminoethyl) methacrylate, methyl methacrylate) 1:2:1

The tablets were obtained as per the method given in Example 1 above.

The tablets thus obtained were subjected to in vitro dissolution testing using United States Pharmacopoeia Type I dissolution apparatus at 100 rpm. The dissolution medium used was 2000 ml of purified water with 0.1% hydroxypropyl methylcellulose. The results obtained are recorded in Table 9 below.

TABLE 9

| Time (hours) | % drug released |
|---|---|
| 1 | 22 |
| 3 | 42 |
| 6 | 57 |
| 12 | 72 |
| 16 | 78 |
| 24 | 84 |

### EXAMPLE 6

The oral controlled release tablets of carbamazepine were prepared as per Table 10 below.

TABLE 10

| Ingredients | Quantity (mg/tablet) | Percent (%) by weight |
|---|---|---|
| Carbamazepine (Milled) | 300.00 | 55.61 |
| Carbamazepine | 100.00 | 18.53 |
| (Micronised) | | |
| Ethyl Cellulose (Ethocel std | 20.00 | 3.71 |
| 45 premium) | | |
| Microcrystalline Cellulose | 60.0 | 11.12 |
| (Avicel PH101) | | |
| Starch | 4.00 | 0.74 |
| Polyvinylpyrrolidone | 15.00 | 2.78 |
| (PVP K30) | | |
| Croscarmellose Sodium | 15.00 | 2.78 |
| (Ac-di-sol) | | |
| Colloidal Silicon dioxide | 5.00 | 0.93 |
| Talc | 6.00 | 1.11 |
| Magnesium Stearate | 5.00 | 0.93 |
| Coat | | |
| Eudragit E 100* | 2.50 | 0.46 |
| Talc | 3.15 | 0.58 |
| Magnesium stearate | 1.00 | 0.18 |
| Titanium dioxide | 2.00 | 0.37 |
| Red oxide of Iron | 0.10 | 0.018 |
| Iron oxide yellow | 0.25 | 0.046 |
| Polyethylene glycol 6000 | 0.50 | 0.093 |

*-poly (butyl methacrylate, (2-dimethyl aminoethyl) methacrylate, methyl methacrylate) 1:2:1

The tablets were obtained as per the method given in Example 1 above.

The tablets thus obtained were subjected to in vitro dissolution testing using United States Pharmacopoeia Type I dissolution apparatus at 100 rpm. The dissolution medium used was 2000 ml of purified water with 0.1% hydroxypropyl methylcellulose. The results obtained are recorded in Table 11 below.

TABLE 11

| Time (hours) | % drug released |
|---|---|
| 1 | 32 |
| 3 | 55 |
| 6 | 71 |
| 12 | 87 |
| 16 | 92 |
| 24 | 97 |

### EXAMPLE 7

The oral controlled release tablets of carbamazepine were prepared in strengths of 100 mg, 200 mg, 300 mg and 400 mg as per Table 10 below.

Table 10

TABLE 10

| | Quantity (mg/tablet) | | | |
|---|---|---|---|---|
| | Strength | | | |
| | 100 mg | 200 mg | 300 mg | 400 mg |
| Ingredients | tablet | tablet | tablet | tablet |
| Carbamazepine (milled) | 75.0 | 150.0 | 225.0 | 300.0 |
| Carbamazepine (micronised) | 25.0 | 50.0 | 75.0 | 100.0 |
| Ethyl cellulose, std 45 premium | 5.00 | 10.00 | 15.00 | 20.00 |
| Microcrystalline cellulose, Avicel | 15.25 | 30.50 | 45.75 | 61.00 |
| PH 101 | | | | |
| Corn starch | 1.00 | 2.00 | 3.00 | 4.00 |
| Polyvinylpyrrolidone (PVP K-30) | 3.50 | 7.00 | 10.50 | 14.00 |
| Croscarmellose sodium | 3.75 | 7.50 | 11.25 | 15.00 |
| Talc | 1.50 | 3.00 | 4.50 | 6.00 |
| Colloidal silicon dioxide | 1.25 | 2.50 | 3.75 | 5.00 |
| Magnesium stearate | 1.25 | 2.50 | 3.75 | 5.00 |
| Coat- | | | | |
| Eudragit E-100* | 1.08 | 2.165 | 3.25 | 4.33 |
| Talc | 1.37 | 2.73 | 4.09 | 5.46 |
| Magnesium stearate | 0.43 | 0.87 | 1.29 | 1.73 |
| Titanium dioxide | 0.86 | 1.72 | 2.59 | 3.45 |
| Red oxide of Iron | 0.016 | 0.032 | 0.048 | 0.064 |
| Polyethylene glycol (PEG 4000) | 0.217 | 0.433 | 0.65 | 0.866 |

*-poly (butyl methacrylate, (2-dimethyl aminoethyl) methacrylate, methyl methacrylate) 1:2:1

All ingredients were passed through a standard ASTM sieve. Carbamazepine, milled and micronised, was mixed intimately with microcrystalline cellulose, starch, PVP K-30 and a part of ethyl cellulose to obtain a powder blend. This powder blend was mixed with a part of the croscarmellose sodium. The mixture was then granulated using a binder solution containing the remaining amount of ethyl cellulose. The granules obtained were dried and then lubricated with a mixture of talc, colloidal silicon dioxide, magnesium stearate and the remaining amount of croscarmellose sodium. The lubricated granules were then compressed to obtain the tablets. A coating solution comprising Eudragit E100, talc, magnesium stearate, titanium dioxide, red oxide of iron and PEG 4000 was used to coat the tablets by conventional means.

### EXAMPLE 8

The bioavailability of the oral controlled drug delivery system for carbamazepine (400 mg tablet of Example 7) and that of marketed carbamazepine controlled drug delivery systems were studied. A single dose, open label, randomized, comparative, two-way crossover study was carried out for the same. Tegretol® XR 400 mg tablets (Novartis, Lot No. 560E0602) were used as the reference standard.

The pharmacokinetic assessment was based on the plasma levels of carbamazepine measured by blood sampling. Blood samples were obtained before dosing and at the following times after administration of both the reference and test medications—1, 2, 4, 6, 8, 12, 16, 18, 20, 22, 24, 28, 32, 36, 48, 72, 96, 120, 144 and 192 hours.

Twelve healthy male volunteers were enrolled for the study and all of them completed the two-way crossover study. The subjects were fasted overnight before dosing and for 4 hours thereafter. Drinking water was prohibited 2 hours before dosing and 2 hours thereafter, but was allowed ad lib at all other times. Standard meals were provided at 4, 6 and 13 hours after dosing and at appropriate times thereafter. Meal plans were identical for both the periods.

Subjects received a single tablet of carbamazepine (400 mg, Example 7) with 240 ml of water at ambient temperature after the overnight fast, as the test medication, while a single tablet of Tegretol® XR 400 mg (Novartis) was administered as the reference medication. A 21-day wash period was allowed between the doses.

The plasma concentration of carbamazepine was determined for samples collected at different time points and averaged over the twelve volunteers. The data is given in Table 11 below.

TABLE 11

| | Plasma concentration (ng/ml) (Mean ± SD) | |
|---|---|---|
| | Carbamazepine tablet | Tegretol ® XR 400 mg |
| Time (hrs) | (400 mg tablet, Example 7) | (Novartis) |
| 0 | 0 | 0 |
| 1.0 | 0.78 | 0.28 |
| 2.0 | 1.77 | 0.89 |
| 4.0 | 2.91 | 1.89 |
| 6.0 | 3.14 | 2.19 |
| 8.0 | 3.37 | 2.54 |
| 12.0 | 3.80 | 3.27 |
| 16.0 | 3.75 | 3.44 |
| 18.0 | 3.79 | 3.51 |
| 20.0 | 3.91 | 3.57 |
| 22.0 | 3.92 | 3.65 |
| 24.0 | 3.92 | 3.72 |
| 28.0 | 4.02 | 3.68 |
| 32.0 | 3.72 | 3.41 |
| 36.0 | 3.67 | 3.38 |
| 48.0 | 3.14 | 2.82 |
| 72.0 | 2.24 | 2.10 |
| 96.0 | 1.57 | 1.50 |
| 120.0 | 1.13 | 1.07 |
| 144.0 | 0.81 | 0.80 |
| 192.0 | 0.43 | 0.43 |

The composition of the present invention was found to be bioequivalent to Tegretol® XR, an osmotic system commercially available in the United States of America, that releases carbamazepine in a controlled zero order manner.

1. An oral controlled drug delivery system comprising carbamazepine and one or more hydrophobic polymers in homogenous admixture, wherein the system does not comprise any means capable of preventing the conversion of carbamazepine to its dihydrate form.

2. An oral controlled drug delivery system as claimed in claim 1, wherein the carbamazepine is crystalline or amorphous.

3. An oral controlled drug delivery system as claimed in claim 1, wherein the carbamazepine is anhydrous or a hydrate.

4. An oral controlled drug delivery system as claimed in claim 1, wherein the carbamazepine is crystalline anhydrous carbamazepine having a particle size such that at least 90% of the particles are below 50 microns.

5. An oral controlled drug delivery system as claimed in claim 1, wherein the hydrophobic polymer is a cellulose derivative selected from the group comprising ethylcellulose, cellulose acetate, cellulose acetate butyrate, cellulose acetate propionate, and mixtures thereof.

6. An oral controlled drug delivery system as claimed in claim 5, wherein the ethylcellulose is selected such that the viscosity of a 5% solution of ethylcellulose in a mixture of 80% toluene and 20% ethanol is in the range of about 40 mPas to about 60 mPas.

7. An oral controlled drug delivery system as claimed in claim 6, wherein the ethylcellulose is used in an amount ranging from about 2% to about 10% by weight of the system.

8. An oral controlled drug delivery system as claimed in claim 7, wherein the ethylcellulose is used in an amount ranging from about 2% to about 5% by weight of the system.

9. An oral controlled drug delivery system as claimed in claim 5 further comprising a hydrophilic polymer that does not prevent conversion of the carbamazepine to its dihydrate form upon contact with water.

10. An oral controlled drug delivery system as claimed in claim 9, wherein the hydrophilic polymer used is a vinyl pyrrolidone polymer.

11. An oral controlled drug delivery system as claimed in claim 10, wherein the vinyl pyrrolidone polymer used has an approximate molecular weight of 50,000 Daltons.

12. An oral controlled drug delivery system as claimed in claim 10, wherein the vinyl pyrrolidone polymer is used in an amount ranging from about 1% to about 5% by weight of the system.

13. An oral controlled drug delivery system as claimed in claim 1, wherein the system comprises tablets that disintegrate in gastric fluids.

14. An oral controlled drug delivery system as claimed in claim 13, further comprising a wicking agent.

15. An oral controlled drug delivery system as claimed in claim 14, wherein microcrystalline cellulose is used as the wicking agent.

16. An oral controlled drug delivery system as claimed in claim 15, wherein the microcrystalline cellulose is used in an amount ranging from about 2% to about 20% by weight of the system.

17. An oral controlled drug delivery system comprising:
(a) crystalline anhydrous carbamazepine having a particle size distribution such that at least 90% of the particles are below 50 microns, in an amount ranging from about 60% to about 85% by weight of the system;
(b) ethylcellulose, selected such that a 5% solution of the same in a mixture of toluene and ethanol has a viscosity of 40-60 mPas, in an amount ranging from about 3% to about 10% by weight of the system;
(c) vinyl pyrrolidone polymer having an approximate molecular weight of 50,000, in an amount ranging from about 1% to about 5% by weight of the system;
(d) microcrystalline cellulose in an amount ranging from about 5% to about 25% by weight of the system;
(e) starch, in an amount ranging from about 0.5% to about 2% by weight of the system; and
(f) croscarmellose sodium in an amount ranging from about 0.5% to about 5% by weight of the system.

18. An oral controlled drug delivery system as claimed in claim 1, wherein the system is bioequivalent to marketed carbamazepine controlled drug delivery systems that release carbamazepine in a controlled zero order manner.

19. An oral controlled drug delivery system as claimed in claim 1 wherein the system is bioequivalent upon oral administration to an oral osmotic controlled zero-order drug delivery system commercially available in the United States of America.

20. An oral controlled drug delivery system as claimed in claim 1 comprising 200 mg of carbamazepine, wherein the system upon oral administration to healthy male volunteers gives a plasma concentration versus time profile with mean plasma concentration lying in the concentration ranges as given below—

| Time (hours) | Plasma concentration range (μg/ml) |
|---|---|
| 8.0 | 1.1 -2.0 |
| 20.0 | 1.4 -2.5 |
| 48.0 | 1.0 -1.8 |
| 120.0 | 0.3 -0.7 |

21. An oral controlled drug delivery system as claimed in claim 1 comprising 400 mg of carbamazepine, wherein the system upon oral administration to healthy male volunteers gives a plasma concentration versus time profile with mean plasma concentration lying in the concentration ranges as given below—

| Time (hours) | Plasma concentration range (μg/ml) |
|---|---|
| 8.0 | 0.9 -3.3 |
| 20.0 | 2.4 -4.4 |
| 48.0 | 2.0 -3.4 |
| 120.0 | 0.6 -1.0 |

22. An oral controlled drug delivery system as claimed in claim 1 wherein the system in a single dose two-way crossover fasted state bioavailability study provides area under the plasma concentration—time curve (AUC) which is comparable to that provided by the oral osmotic controlled zero-order drug delivery system commercially available in the United States of America.

23. An oral controlled drug delivery system as claimed in claim 1 wherein the system in a single dose two-way crossover fasted state bioavailability study provides peak plasma levels that are comparable to those provided by the oral osmotic controlled zero-order drug delivery system commercially available in the United States of America.

24. An oral controlled drug delivery system as claimed in claim 22 wherein the system is bioequivalent with the oral osmotic controlled zero-order drug delivery system commercially available in the United States of America.
